(19) United States
(12) Patent Application Publication
(10) Pub. No.: US 2006/0141498 A1
(43) Pub. Date: Jun. 29, 2006

(54) Methods for fragmenting nucleic acid

(75) Inventors: Kai Wu, Mountain View, CA (US); Charles Garrett Miyada, San Jose, CA (US); Thong Nguyen, San Jose, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/273,964

(22) Filed: Nov. 14, 2005

Related U.S. Application Data
(63) Continuation-in-part of application No. 10/951,983, filed on Sep. 27, 2004.
(60) Provisional application No. 60/506,697, filed on Sep. 25, 2003.
(60) Provisional application No. 60/512,569, filed on Oct. 15, 2003.
(60) Provisional application No. 60/512,301, filed on Oct. 16, 2003.
(60) Provisional application No. 60/514,872, filed on Oct. 28, 2003.
(60) Provisional application No. 60/547,915, filed on Feb. 25, 2004.
(60) Provisional application No. 60/627,053, filed on Nov. 12, 2004.
(60) Provisional application No. 60/683,127, filed on May 19, 2005.

(51) Int. Cl. C12Q 1/68; C12P 19/34; C12N 9/22

(52) U.S. Cl. 435/6; 435/91.2; 435/199

(57) ABSTRACT

Methods for using an apurinic/apyrimidinic endonuclease, capable of cleaving both single- and double-stranded cDNA, for fragmentation and labeling of single stranded or double stranded DNA molecules are provided. Amplification methods that generate single-stranded amplified cDNA are also disclosed. In the subject methods AP sites in a population of nucleic acids are cleaved by an AP endonuclease that is active on both double and single stranded DNA. Fragments may be end labeled. In preferred embodiments APE 1 is used. The methods may be used in a variety of applications where end-labeling single or double stranded DNA is desired.

### RELATED APPLICATIONS

This application is a continuation in part of U.S. application Ser. No. 10/951,983 which claims priority to U.S. Provisional Application Ser. No. 60/506,697 filed on Sep. 25, 2003, U.S. Provisional Application Ser. No. 60/512,569 filed on Oct. 15, 2003, U.S. Provisional Application Ser. No. 60/512,301 filed on Oct. 16, 2003, U.S. Provisional Application Ser. No. 60/514,872 filed on Oct. 28, 2003 and U.S. Provisional Application Ser. No. 60/547,915 filed on Feb. 25, 2004. This application also claims priority to U.S. Provisional Application Ser. No. 60/627,053 filed on Nov. 12, 2004 and U.S. Provisional Application Ser. No. 60/683,127 filed on May 19, 2005. Each cited patent application is incorporated herein by reference in its entirety for all purposes.

### FIELD OF THE INVENTION

The field of this invention is nucleic acids, particularly nucleic acid fragmentation and labeling techniques.

### BACKGROUND OF THE INVENTION

Nucleic acid hybridization methods often benefit from fragmentation and labeling of the target nucleic acids prior to hybridization. The conventional method for fragmentation of DNA molecules utilizes DNase I to digest the DNA molecules, which is a controlled enzymatic process with no specific sequence preference. The products of DNase I digestion are fragments with 3′-OH termini ready for terminal labeling by terminal transferase (TdT). The process of DNase I digestion is difficult to modulate to avoid over or under digestion which produces fragments with less than desired length. There remains a need in the art for methods for reproducibly and efficiently fragmenting nucleic acids for hybridization to microarrays.

### SUMMARY OF THE INVENTION

Methods are disclosed for preparing amplified, fragmented, end labeled cDNA for hybridization to an array. The cDNA population for fragmentation is preferably single stranded, but may also be double stranded or a mixture of both single stranded and double stranded. In many embodiments the cDNA is part of a complex nucleic acid sample. Fragmented cDNA may be end labeled at the 3′ or 5′ end with a detectable label, for example, a biotinylated nucleotide.

In a particularly preferred aspect an RNA sample is subjected to two cycles of amplification to generate single-stranded cDNA that is sense in orientation. The first cycle includes ds-cDNA synthesis followed by in vitro transcription of antisense cRNA. The second cycle includes synthesis of single-strand sense cDNA with incorporation of uracil into the cDNA. The uracil containing cDNA is fragmented using an AP endonuclease.

In one embodiment the cDNA has uracil incorporated at a ratio of about 1:4 (UTP to TTP), 1:3, 1:5, 1:6, 1:10, 1:15, or 1:20. The ratio of UTP to TTP in the cDNA determines the average size of the resulting fragments, more uracil incorporated results in smaller average fragment size. In one embodiment the fragments average about 40 to 70 bases in length and the majority of the fragments are between 40 and 150 bases in length.

The fragments are preferably analyzed by hybridization to an array of nucleic acid probes. In one embodiment the array includes a solid support with different sequence probes attached at known locations. In another embodiment the probes of the array are attached to beads or microparticles. The beads or microparticles may be marked with an encoding system such as a tag, a barcode or an optical signature so that the sequence of the probe on a given bead is known or can be determined. Beads may be in solution or may be associated at locations in an array of beads.

In one embodiment the uracil containing DNA is treated with UDG to generate abasic sites and then with an AP endonuclease that has cleavage activity on single stranded DNA or both single and double stranded DNA. In preferred embodiments the AP endonuclease is APE 1 or a variant of APE 1, for example, a variant that is at least about 90% homologous to human APE 1.

In one embodiment an oligonucleotide that may be used to monitor the efficiency of the fragmentation reaction may be included in the sample before or during the steps prior to fragmentation. The control oligo may have a 5′ first region and a 3′ second region that are separated by a site that can be cleaved by an AP endonuclease. In some embodiments the first and second regions are separated by at least one uracil so the oligo can be fragmented by UDG and APE 1 treatment. In some aspects there are between 2 and 4 uracils. The array preferably includes probes for the 5′ region and may also include probes for the 3′ region. The 3′ end of the control oligo preferably is blocked from extension and labeling. If the oligo is fragmented a new 3′ end that is compatible with end labeling is generated so that the first region can be labeled only after fragmentation. The probes for the first region should detect hybridization while the probes for the second region should not have signal above background.

### BRIEF DESCRIPTION OF THE DRAWINGS

FIG. 1 is a schematic of a method of generating an amplicon containing labeled single-stranded sense cDNA fragments from an RNA sample.

FIG. 2 is a schematic of a method of generating an amplicon containing labeled double-stranded cDNA fragments from an RNA sample.

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947 and 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989), Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996, 6,386,749, and 6,391,623 each of which is incorporated herein by reference.

The present invention also contemplates signal detection of hybridization between ligands in certain preferred embodiments. See U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625, in U.S. Ser. No. 10/389,194, and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication Number 20020183936), Ser. Nos. 10/065,856, 10/065,868, 10/328,818, 10/328,872, 10/423,403, and 60/482,389.

b) Definitions

The term “admixture” refers to the phenomenon of gene flow between populations resulting from migration. Admixture can create linkage disequilibrium (LD).

The term “allele’ as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules, libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer. Avidin, biotin, antibodies, antibody fragments, etc., for example, are also biomonomers.

The term “biopolymer” or sometimes “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, and synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors in a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, more preferably at least about 90% complementary. See, M. Kanehisa, Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml and acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al., Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture of DNA or RNA, for example, total cellular RNA or DNA or nucleic acid.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “mixed population” or “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or “mRNA transcripts” as used herein, includes, but is not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or “array” as used herein refers to an intentionally created collection of nucleic acids which can be prepared either synthetically or biosynthetically and screened for biological activity in a variety of different formats (for example, libraries of soluble molecules and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms (RFLPs), variable number of tandem repeats (VNTRs), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “ligand receptor pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates. The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. A “probe target pair” is formed when two macromolecules have combined through molecular recognition to form a complex.

c) Description

Methods are provided for amplification of nucleic acids to generate amplified DNA, fragmentation of the DNA and labeling of the fragments. The fragmented, labeled DNA is suitable for a variety of analyses methods, including hybridization to arrays of nucleic acid probes bound to one or more solid supports. Methods for fragmentation and labeling of nucleic acids for microarray analysis are also disclosed in U.S. Patent Publication No. 20050123956. Methods for amplifying nucleic acid samples that may be fragmented and labeled using methods disclosed herein are disclosed, for example, in U.S. Patent Publication No. 20050106591.

In one aspect methods are provided for using an apurinic/apyrimidinic endonuclease that is active on single-stranded DNA to fragment cDNA. The fragments are then end-labeled. In the subject method, deoxyuridine (dUTP) is incorporated into a sample DNA molecule during first strand cDNA synthesis, the template RNA is removed and the single-stranded cDNA is fragmented using a UDG activity and an AP endonuclease activity. In a preferred embodiment the AP endonuclease is a human AP endonuclease, for example, APE 1, which cleaves abasic sites in both single and double stranded DNA. The fragmentation process produces DNA fragments within a certain range of length than can be subsequently labeled at the 3′-termini, for example, with a biotinylated compound using a TdT activity.

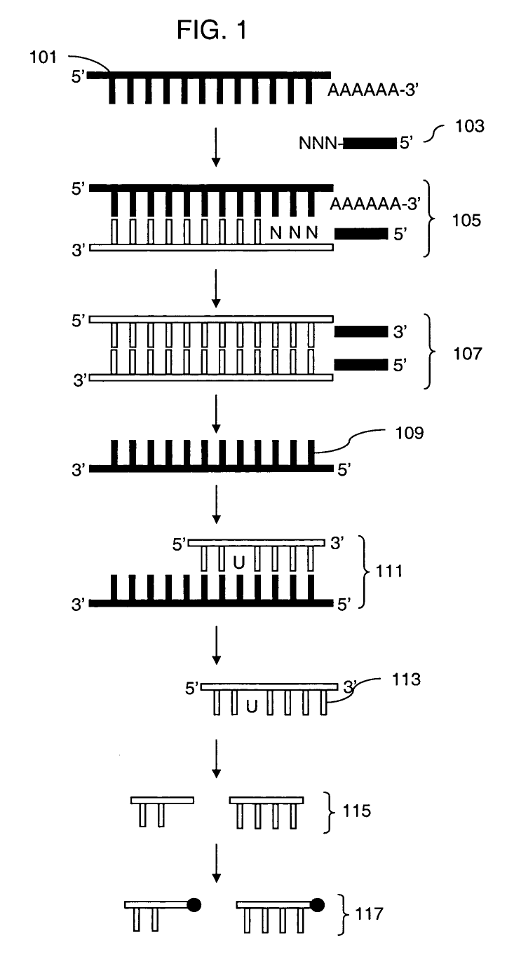

FIG. 1 shows a schematic of a preferred embodiment. A sample containing RNA (101) is reverse transcribed using T7-(N)₆ primers (103) to generate an RNA:DNA hybrid (105). Second strand cDNA synthesis generates a double-stranded cDNA with a T7 promoter (107). The double-stranded cDNA is used as template in an in vitro transcription reaction resulting in the production of antisense cRNA (109) which is preferably unlabeled. The antisense cRNA is used as template in a reverse transcription reaction primed by random primers and in the presence of a mixture of dGTP, dCTP, dTTP, dATP and dUTP, generating cDNA containing uracil in RNA:DNA hybrids (111). The cRNA may be removed or hydrolyzed, for example, by RNase H treatment, leaving single-stranded uracil containing cDNA (113). The cDNA (113) may be cleaned up and mixed with UDG and APE 1 to generate cDNA fragments (115). The cDNA fragments may be end labeled using TdT and DLR. In a particularly preferred embodiment the RNA sample (101) is total RNA that has been subjected to one or more steps for reduction of ribosomal RNA, for example, by treatment with RIBOMINUS from Invitrogen.

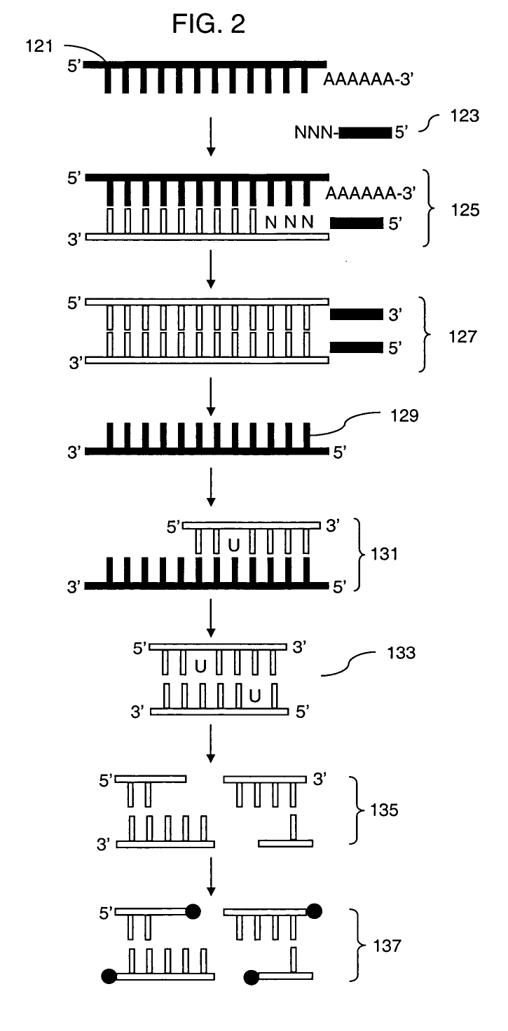

In another embodiment, shown in FIG. 2, sense and antisense cDNA is generated and double stranded cDNA is fragmented by an AP endonuclease. A sample containing RNA (121) is reverse transcribed using T7-(N)₆ primers (123) to generate an RNA:DNA hybrid (125). Second strand cDNA synthesis generates a double-stranded cDNA with a T7 promoter (127). The double-stranded cDNA is used as template in an in vitro transcription reaction resulting in the production of antisense cRNA (129) which is preferably unlabled. The antisense cRNA is used as template in a reverse transcription reaction primed by random primers and in the presence of a mixture of dGTP, dCTP, dTTP, dATP and dUTP, generating cDNA containing uracil in RNA:DNA hybrids (131). E. coli DNA polymerase and RNase H are added to generate second strand cDNA, resulting in double-stranded cDNA (133). Both strands of the ds-cDNA contain uracil. UDG and APE1, or another AP endonuclease that cleaves double stranded DNA, are added to fragment the DNA generating double stranded cDNA fragments (135). The fragments are end labeled (137). In preferred aspects E. coli DNA polymerase is used if the desired target is single stranded cDNA, because the enzyme is less prone to spurious copying of the original strand. Where the desired product is double-stranded target polymerases such as Klenow (exo−) may be preferred. Klenow is more prone to creating copies of the original strand.

Methods for using apurinic/apyrimidinic endonuclease for fragmentation and end-labeling of DNA molecules are disclosed. Single or double-stranded nucleic acid molecules may be fragmented and labeled. In a preferred embodiment DNA molecules that may be end-labeled according to the methods are nucleic acids that, once fragmented, have a free 3′ hydroxyl group. The DNA molecules can be any desired chemically and enzymatically synthesized nucleic acid, e.g., a nucleic acid produced in vivo by a cell or by in vitro amplification.

In a preferred embodiment an apurinic/apyrimidinic endonuclease is used to cleave an apyrimidinic site within a DNA molecule to yield a fragment with a certain range of length and a 3′-OH terminus. The 3′-OH terminus may be used for terminal labeling. In some embodiments the apurinic/apyrimidinic endonuclease generates a 3′-phosphate terminus and the phosphate is subsequently removed, for example, by adding phosphatase to the reaction, generating a 3-OH terminus conducive for subsequent terminal labeling. In a preferred embodiment, apurinic/apyrimidinic endonucleases which create a 3′-OH terminus that may be used include, endonuclease V, endonuclease VI, endonuclease VII, human endonuclease II, and the like. In the subject invention, apurinic/apyrimidinic endonucleases which create a 3′-phosphate terminus consist of, but are not limited to endonuclease III, endonuclease VIII, and the like. Any apurinic/apyrimidinic endonuclease involving hydrolytic based cleavage would be appropriate for use with the disclosed methods.

The fragmentation process employed in the subject method begins with creating cleavable fragments. The first step in creating these fragments is the incorporation of an exo-nucleotide (a nucleotide which is generally not found in the sample DNA molecule or nucleic acid) or the incorporation of normal nucleotides that are then converted to exo-nucleotides into a sample DNA molecule or sample nucleic acid. dUTP is an example of an exo-nucleotide because generally it is rarely or found naturally in DNA. Although the triphosphate form of dUTP is present in living organisms as a metabolic intermediate, it is rarely incorporated into DNA. When dUTP is accidentally incorporated into DNA, the resulting deoxyuridine is promptly removed in vivo by normal process, e.g., processes involving the enzyme UDG. Thus, deoxyuridine occurs rarely or never in natural DNA. It is recognized that some organisms may naturally incorporate deoxyuridine into DNA. See U.S. Pat. No. 5,035,996. Normal nucleotides can be converted into exo-nucleotides by converting neighboring pyrimidine or purine residues, i.e. converting neighboring pyrimidine residues in thymidine to create pyrimidines dimmers. See U.S. Pat. Nos. 5,035,996 and 5,683,896.

In a preferred embodiment the DNA to be fragmented is a product amplified from a nucleic acid sample isolated from a biological source. In a preferred embodiment the DNA to be fragmented is an amplification product resulting from amplification of an RNA sample isolated from one or more cells. In a particularly preferred embodiment RNA is isolated from a source, first strand cDNA is generated by reverse transcription with primers comprising a random 3′ sequence and a 5′ RNA polymerase promoter sequence, for example, random hexamer-T7 primers, the first strand cDNA is used to generate second strand cDNA resulting in dsDNA with an RNA polymerase promoter, and unlabeled cRNA is transcribed by IVT. The antisense RNA (cRNA) product is the output of the first cycle of amplification and is used as the starting template for a second cycle of amplification. In the second cycle first strand cDNA is synthesized using the cRNA as template for an extension reaction primed by random primers. During this second cycle of first strand cDNA synthesis dUTP is present and is incorporated into the cDNA. The cRNA may then be hydrolyzed, for example, by treatment with RNase H and the sense stranded cDNA can be cleaned-up. The cDNA may then be treated with UDG and APE 1 to fragment and then fragments may be end labeled using TdT and a labeled nucleotide such as Affymetrix′ DNA Labeling Reagent. The labeled cDNA may then be hybridized to an array.

In another aspect the second cycle of amplification includes an optional step of second strand cDNA synthesis and the products are double-stranded cDNA In the second round of cDNA synthesis uracil may be incorporated into the first strand cDNA or the second strand cDNA or both. For a detailed example see Example 3 below.

The amount of starting material may be, for example, about 10 or 100 to 500 ng of total RNA. In some aspects less than 10 ng total RNA may be used as starting material. If the total RNA is subjected to a complexity reduction step, for example, depletion of rRNA or globin mRNA or enrichment of mRNA, less RNA may be used as starting material. Preferably about 5 or 10 to 100 μg and more preferably about 20 μg of labeled target may be used for hybridization to one array. In some embodiments total RNA may be treated to remove selected sequences that may interfere with analysis, for example, ribosomal RNA (rRNA) may be removed prior to amplification. Many methods of removing rRNA are known to one of skill in the art, for example, see U.S. Pat. No. 6,613,516 which describes hybridization of oligonucleotides that are complementary to ribosomal RNA to the ribosomal RNA, optionally extending the oligonucleotides and cleaving the rRNA with RNaseH activity. Another method of depleting rRNA, or another RNA that is not of interest, that may be used is to incubate the total RNA with a solid support (for example, beads, membrane or resin) comprising oligonucleotides that are complementary to rRNA sequences to allow rRNA to bind to the solid support. The bound rRNA may then be separated from the remaining total RNA that is in solution. In another embodiment globin mRNAs may be removed or depleted. Globin mRNAs are present in very high amounts in RNA isolated from blood and can interfere with detection of other mRNAs. Globin mRNAs may be removed, for example, by depletion using a solid support that has globin complementary oligonucleotides associated or attached as described above for rRNA, by hybridization of blocking oligonucleotides to the globin mRNA, the blocking oligos may prevent amplification of globin mRNAs by blocking reverse transcription of the globin mRNAs, or the globin mRNA may be depleted by hybridization of globin complementary oligos, optionally extension of the oligos and cleavage of the mRNA with RNase H. In some embodiments the oligonucleotides used contain one or more modified nucleotides, for example, peptide nucleic acids (PNAs) or locked nucleic acids (LNAs). For additional description of these methods see, for example, U.S. Pat. No. 6,613,516 and U.S. patent application Ser. No. 10/684,205. When rRNA is depleted less of the final product may be hybridized to a single array, for example, in one embodiment without rRNA depletion 20 μg is hybridized to an array and with rRNA depletion 5 μg of the labeled, fragmented cDNA is hybridized to the array.

In a preferred embodiment dUTP is incorporated into the sample DNA molecule or sample nucleic acid. dUTP can be incorporated via a reverse transcription reaction, preferably a specific ratio of dTTP to dUTP is used. This ratio of dTTP to dUTP is selected to generate DNA fragments of a pre-determined size range. In one preferred embodiment the fragment lengths show a peak at 40 to 50 bases with about 90% or more of the fragmented material being between 25 and 150 bases in length. In a preferred embodiment of the invention, the reverse transcription reaction is run so that the total RNA is reverse transcribed with dNTPs at a final concentration of about 0.5 mM. See U.S. Pat. Nos. 5,035,996 and 5,683,896

Next, the sample DNA molecules or nucleic acids are processed in a reaction comprising DNA glycosylase to create an abasic site. DNA glycosylases release bases from DNA by cleaving the glycosidic bond between the deoxyribose of the DNA sugar-phosphate backbone and the base. DNA glycosylases are capable of releasing, including but not limited to, cytosine bases from ssDNA and dsDNA, thymine bases from ssDNA and dsDNA, and uracil bases from ssDNA or dsDNA. DNA glycosylases are base specific. Therefore, the appropriate DNA glycosylase is dependent upon which base was incorporated into the sample DNA molecule or sample nucleic acid. See U.S. Pat. No. 6,713,294.

In the preferred embodiment of the subject invention, UDG specifically recognizes uracil and removes it by hydrolyzing the N—C1′ glycosylic bond linking the uracil base to the deoxyribose sugar. The loss of the uracil creates an abasic site (also known as an AP site or apurinic/apyrimidinic site) in the DNA. An abasic site is a major form of DNA damage resulting from the hydrolysis of the N-glycosylic bond between a 2-deoxyribose residue and a nitrogenous base. This site can be generated spontaneously or as described above, via UDG catalyzed hydrolysis See Marenstein et al. (2004) DNA Repair 3:527-533.

Subsequent treatment of the sample DNA molecule or sample nucleic acid with alkaline solutions or enzymes, such as but not limited to apurinic/apyrimidinic endonucleases, will cause controlled breaks in the DNA at the abasic site. See U.S. Pat. No. 6,713,294. The abasic site can be cleaved by physical or enzymatic means. While high temperature or high pH induced hydrolysis can generate cleavage at abasic sites, the resulting 3′ termini of the cleavage may not be a substrate for labeling by TdT. An apurinic/apyrimidinic endonuclease can cleave the DNA molecule or nucleic acid at the site of the dU residue yielding fragments possessing a 3′-OH termini, thus allowing for subsequent terminal labeling. One such apurinic/apyrimidinic endonuclease is E. coli Endo IV which catalyzes the formation of single-strand breaks at apurinic and apyrimidinic sites within a double-stranded DNA to yield 3′-OH termini suitable for terminal labeling. E. coli Endo IV may also be used to remove 3′ blocking groups (e.g. 3′-phosphoglycolate and 3′-phosphate) from damaged ends of double-stranded DNA. See Levin, J. D., J. Biol. Chem., 263:8066-8071 (1988) and Ljungquist, et al., J. Biol. Chem., 252:2808-2814 (1977).

In a preferred embodiment the AP endonuclease is human APE 1 or a variant thereof. Human APE 1, unlike E. coli Endo IV, is capable of cleaving either single-stranded or double-stranded substrate at AP sites. APE 1 is also known as Hap1, Apex, and Ref1 and can be utilized in conjugation with UDG to perform cleavage at dU incorporation sites in single-strand and double strand DNA. APE 1 is an enzyme of the base excision repair pathway which catalyzes endonucleolytic cleavage immediately 5′ to abasic sites. See Marenstein supra. Additional information about APE 1 may be found in Robson, C. N. and Hickson, D. I. (1991) Nucl. Acids Res., 19, 5519-5523, Vidal, A. E. (2001) EMBO J., 20, 6530-6539, Demple, B. et al. (1991) Proc. Natl. Acad. Sci. USA, 88, 11450-11454, Barzilay, G. et al. (1995) Nucl. Acids Res., 23, 1544-1550, Barzilay, G. et al. (1995) Nature Struc. Biol., 2, 451-468, Wilson, D. M. III et al. (1995) J. Biol. Chem., 270, 16002-16007, Gorman, M. A. et al (1997) EMBO J., 16, 6548-6558, Xanthoudakis, S. et al. (1992) EMBO J., 11, 3323-3335, Walker, L. J. et al. (1993) Mol. Cell. Biol., 13, 5370-5376, and Flaherty, D. M. (2001) Am. J. Respir. Cell. Mol. Biol., 25, 664-667, each of which is incorporated herein by reference in its entirety for all purposes.

APE 1 acts on both dsDNA and ssDNA. The catalytic efficiency of the cleavage of ssDNA is approximately 20-fold less than the activity against AP sites in dsDNA. Catalysis is Mg²⁺ dependent. Unlike the activity of APE 1 against AP sites in dsDNA, it does not display product inhibition when acting on an AP site in ssDNA. One unit of APE 1 is defined by the supplier (New England Biolabs) as the amount of enzyme required to cleave 20 pmol of a 34 mer oligonucleotide duplex containing a single AP site in a total reaction volume of 10 μl in 1 hour at 37° C.

The amount of dU incorporation may be regulated to determine the average length of fragments after UDG/APE 1 treatment. The ratio of dUTP to dTTP may be, for example, about 1 to 4, or about 1 to 5, 1 to 6, 1 to 10 or 1 to 20. One of skill in the art will appreciate that varying the ratio of dUTP to dTTP will result in variation of the amount of dUTP incorporated and result in variation in the average size of fragments. The higher the ratio of dUTP to dTTP the more uracil incorporated and the shorter the average size of the fragments. In a preferred embodiment the fragments are on average about 40 to 50 nucleotides in length, with more than 90% of the fragments being between 25 and 150 bases in length. In another embodiment the fragments are on average between 25 and 50, 40 and 70, 40 and 80, 50 and 100 or 30 to 150 bases or base pairs in length. Longer or shorter fragment sizes may also be achieved by varying the reaction conditions.

In some aspects kits are provided for obtaining amplified cDNA from RNA and fragmenting and labeling the cDNA for hybridization. In one aspect a fragmentation and labeling kit is provided. The kit may include, for example, cDNA fragmentation buffer, UDG, APE 1, TdT, TdT buffer, a labeled nucleotide, for example, DLR1a. The components are preferably provided in a concentrated form, for example, buffers may be provided in the kit as 10× or 5× stocks. The UDG is preferably provided at about 10 U/μl and the APE 1 is preferably about 1000 U/μl. Higher concentrations of APE 1 are used for fragmentation of single-stranded cDNA target.

In another aspect a kit for generating amplified sense strand cDNA from total RNA may be provided. The kit may include T7-(N)₆ primers at about 2.5 μg/μl, 5× first strand cDNA synthesis buffer, 100 mM DTT, 10 mM dNTP mix, RNase inhibitor (40 U/μl), MgCl₂ (1 M), a reverse transcriptase, such as SuperScript II, a DNA polymerase, such as DNA Pol 1, a random primer solution (3 μg/μl), RNase H (2 U/μl), water and a DNTP+dUTP mix. The kit may also include reagents for in vitro transcription including an NTP mix, 10× IVT buffer, IVT enzyme mix and IVT controls. The cDNA synthesis reagents may be organized in a first box as a first sub kit and the IVT reagents may be organized in a second box as a second sub kit. The first and second boxes may be packaged together in a third box.

When utilizing the above fragmentation method with APE 1 for single-stranded cleavage of cDNA, the RNA strand may be digested by either alkaline hydrolysis or enzymatic digestion. For example, the alkaline hydrolysis would occur in alkaline conditions at 55-75° C. for 20-40 minutes. Another example would be performing the enzymatic digestion with RNase H, or an enzyme with similar properties, at 27-47° C. for 20-60 minutes. The remaining DNA strand may then be purified before fragmentation. When utilizing the above method for double-stranded cleavage, a second strand DNA synthesis is performed and the double-stranded DNA is purified before fragmentation. The fragmentation of either single or double-stranded DNA is performed in the presence of UDG and APE 1 and appropriate buffering conditions for APE 1. The reaction is incubated at 27-47° C. for 1-2 hours. The enzymes are heat inactivated at about 93° C. for about 1 minute.

In a preferred embodiment fragmented DNA is labeled. Labeling in one embodiment is by end labeling, for example, labeling of 3′ hydroxyls using TdT. The fragments are incubated in a reaction with TdT, buffer, CoCl₂, and DNA labeling reagent (a biotinylated nucleotide analogue) or any other suitable label. The reaction may be incubated at 27-47° C. for about 1 hour. Preferably more than 80% of the fragments are labeled.

After the fragments have been end-labeled, the product of labeled DNA fragment may be hybridized to a microarray. Examples of microarrays that may be used for analysis are available from Affymetrix, Inc. and include, for example, the HG-U133A 2.0 array.

In one embodiment a control oligonucleotide may be used to monitor the apurinic/apyrimidinic mediated process. A control oligonucleotide for assaying APE 1 mediated fragmentation may include, for example, sequences homologous to those of array control probe set(s). The structure of the control oligonucleotide may be 5′-ProbeA-dU-ProbeB-modified-3′ where Probe A is complementary to a first probe on the array and Probe B is complementary to a second probe on the array. One example for a possible control oligonucleotide to be used in monitoring an APE 1 mediated fragmentation and labeling reaction is: 5′CCCCATGTTCATTGACAAATGTTAAUTGATTCACCGATAAGTACAGCTCGC-3′ (SEQ ID NO. 1). The 5′ portion, CCCCATGTTCATTGACAAATGTTAA (SEQ ID NO. 2), is complementary to a first probe (Probe A) within the AFFX-TRPHX-3_{—}AT probe set on the U133 array. The second portion, 5′ TGATTCACCGATAAGTACAGCTCGC 3′ (SEQ ID NO. 3), is complementary to a second probe (Probe B) within the same probe set. In a preferred embodiment the two sequences are separated by at least one uracil. However, another exo-nucleotide or normal nucleotide converted to an exo-nucleotide could be used. In these embodiments the oligonucleotide may be used to test the function of the DNA glycosylase used in the reaction dependent upon the base used. In another aspect the uracil is replaced with one or more abasic sites. This allows for analysis of the APE 1 cleavage independent of the UDG cleavage.

The 3′ terminal of the control oligonucleotide is preferably modified such that it cannot be extended or labeled. Methods for blocking the 3′ terminal of the control oligonucleotide may include, but are not limited to, the addition of a phosphate, the addition of a modified base such as an amino group, a 3′ deoxy terminator base, a dideoxy base, the addition of a space-linkage, or creating an inverted 3′-3′ linkage. In another embodiment of the control oligonucleotide, dU can be replaced by an apurinic/apyrimidinic base. For assays using double-stranded DNA as substrate, a double-stranded version can be made by annealing the complementary sequence or a substantially complementary sequence to the 5′-Probe A-dU-ProbeB-modified-3′ oligonucleotide.

In microarray applications, the control oligonucleotides may be added along with the sample before the assay. The assay process produces labeled 3′termini for Probe A, but not Probe B thus in the array analysis Probe A would call “Present” and Probe B “Absent”. When the example control oligonucleotide from above is added to the reaction mixture along with the sample, the fragmentation and labeling procedure would yield a biotinylated fragment with the following sequence: (SEQ ID NO: 2) 5′CCCCATGTTCATTGACAAATGTTAA-bio3′, which would hybridize only with Probe A of the AFFX-TRPHX-3_{—}AT probe set, but not with Probe B. The purpose of the B probe set sequence is to provide a negative control as well as other modes of action, i.e. double color labeling in which either the 5′ terminal or 3′ terminal could be pre-labeled with one moiety different from the cleavage/labeling moiety, so that without APE action one should observe labeling of moiety one on A and B probe sets while a different labeling moiety on a different probe set should be observed after the cleavage/labeling.

Related methods of fragmenting are disclosed in U.S. Patent Application Nos. 60/547,915 filed Feb. 25, 2004, 60/512,301 filed Oct. 16, 2003 and 60/550,368 filed Mar. 4, 2004. Each is incorporated herein by reference in its entirety for all purposes and particularly for disclosure related to fragmentation methods using UDG and EndoIV. Other methods of fragmenting are disclosed in U.S. Patent Application Nos. 60/545,417 filed Feb. 17, 2004, 60/589,648 filed Jul. 20, 2004, and 60/616,652 filed Oct. 6, 2004. Each of which is incorporated herein by reference in its entirety for all purposes and particularly for disclosure related to fragmentation methods, including chemical fragmentation methods.

It is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

### EXAMPLE 1

The following steps were performed: (1) incorporating uracil into single-stranded DNA; (2) adding UNG along with APE 1 to cleavage single-stranded substrate containing uracil; (3) 3′-terminal labeling of biotin compounds using TdT; (4) hybridizing labeled fragments with a microarray; and (5) designing control oligonucleotide(s) to assay APE 1 mediated fragmentation/labeling process by microarray hybridization analysis.

First, dUTP was incorporated into a single-stranded cDNA molecule via a reverse transcription reaction at a specific ratio of dTTP to dUTP. Total RNA was reverse transcribed with dNTPs at a final concentration of 0.5 mM. The RNA strand was digested by enzymatic digestion with RNase H at 37° C. for 30 minutes. The remaining cDNA strand was then purified before fragmentation. The fragmentation of the single-stranded cDNA molecule was performed in the presence of UDG and APE 1 under the appropriate buffering conditions for APE 1. The reaction was incubated at 37° C. for 1-2 hours. The enzymes were then inactivated at 93° C. for 1 minute. For end labeling, TdT, buffer, and CoCl₂ along with Affymetrix-proprietary DNA labeling reagent (DLR) with a 0.07 mM final concentration, were added to the fragmentation reaction to end-label the DNA fragments. The reaction was incubated at 37° C. for 1 hour. The end-labeled fragments were hybridized to HG-U133A 2.0 arrays for analysis.

A control oligonucleotide for assaying APE 1 mediated fragmentation consists of sequences homologous to those of array control probe set(s) with structure as following: 5′-Probe A-dU-ProbeB-modified-3′, in which 3′ terminal is modified such that it cannot be extended or labeled and dU could also be replaced by an apurinic/apyrimidinic base. The control oligonucleotide(s) was added along with the sample before the assay. The assay process produced labeled 3′-termini for Probe A but not Probe B, thus in the array analysis Probe A would call “present” and Probe B “Absent.” The results can be seen below in Table 1.

TABLE 1
First Set of APE 1 Experiments

| Experiment | Noise | Scale | | | Average |
|---|---|---|---|---|---|
| Name | (RawQ) | Factor | Background | Present | Signal (all) |
| probe_A | 0.61 | 1 | 27.47 | 65.00% | 74.6 |
| probe_B | 0.62 | 1 | 29.1 | 59.90% | 58.3 |

sRcUAT = ss cDNA, Rnase H, clean, UDG, APE 1 and TDT

### EXAMPLE 2

The method includes the following steps: (1) incorporating dU into double-stranded DNA; (2) adding UNG along with APE 1 to cleavage double-stranded substrate containing dU; (3) 3′-terminal labeling of biotin compounds using TdT; (4) hybridizing labeled fragments with a microarray; and (5) designing control oligonucleotide(s) to assay APE 1 mediated fragmentation/labeling process by microarray.

First, dUTP was incorporated into a double-stranded cDNA molecule via a reverse transcription reaction at a specific ratio between dTTP and dUTP. Total RNA was reverse transcribed with dNTPs at a final concentration of 0.5 mM and subsequently the second strand is synthesized by DNA polymerase. For example, a second strand DNA synthesis may be performed by adding DNA Polymerase I, NDA Ligase, RNase H, and second strand buffer to the first strand reaction. This example embodiment is performed at 16° C. for 2 hours. The double stranded was then purified before fragmentation. The fragmentation of the double-stranded cDNA molecule was performed in the presence of UDG and APE 1 under the appropriate buffering conditions for APE 1. The reaction was incubated at 37° C. for 1-2 hours. The enzymes were then inactivated at 93° C. for 1 minute. Next TdT, buffer, and CoCl₂ along with Affymetrix-proprietary DNA labeling reagent with a 0.07 mM final concentration were added to the fragmentation reaction to end-label the DNA fragments. The reaction was incubated at 37° C. for 1 hour.

Next, the end-labeled fragments were hybridized to Affymetrix human cDNA Test Arrays for analysis. A control oligonucleotide for assaying APE 1 mediated fragmentation consists of sequences homologous to those of array control probe set(s) with structure as following: 5′-Probe A-dU-ProbeB-modified-3′, in which 3′ terminal is modified such that it cannot be extended or labeled and dU could also be replaced by an apurinic/apyrimidinic base. The control oligonucleotide(s) was added along with the sample before the assay. The assay process produced labeled 3′-termini for Probe A but not Probe B, thus in the array analysis Probe A would call “Present” and Probe B “Absent.” The results can be seen below in Table 2.

TABLE 2
Second Set of APE 1 Experiments - using ds cDNA

| Experiment | Noise | Scale | | | Average |
|---|---|---|---|---|---|
| Name | (RawQ) | Factor | Background | Present | Signal (all) |
| APE 120U A | 1.46 | 1 | 65.02 | 62.60% | 25.2 |
| APE 120U B | 1.49 | 1 | 37.27 | 56.20% | 21.3 |
| APE 70U A | 1.47 | 1 | 34.67 | 66.40% | 30.2 |
| APE 70U B | 1.51 | 1 | 36.43 | 65.60% | 29.1 |

### EXAMPLE 3

### cRNA Amplification to Generate ds-cDNA

Step 1. First strand cDNA synthesis: Mix total RNA sample and RP-T7 primer (SEQ ID NO. 4) 5′-GAATTGTAATACGACTCACTATAGGGN₆-3′) (Invitrogen) thoroughly in a 0.2 mL of PCR tube: 3 μL total RNA (˜50 ng) and 2 μL (12.5 pmol/μL) RP-T7 primer. Incubate at 65° C. in thermal cycler for 5 minutes and then at 4° C. for 2 minutes. Then spin down to collect the sample. Prepare the RT_Premix_{—}1 as follows: 2.0 μL 5× first strand buffer, 1.0 μL 0.1 M DTT, 0.5 μL 10 mM dNTP mix, 0.5 μL 40 U/μL RNaseOUT, and 1.0 μL 200 U/μL SuperScript II (Invitrogen) in a total volume of 5 μl. Add 5 μL of the RT_Premix_{—}1 to the denatured RNA and primer mixture to make a final volume of 10 μL. Mix thoroughly, spin down, and incubate at 25° C. for 10 min., at 42° C. for 1 hour, at 70° C. for 10 min. then keep at 4° C. for no longer than 10 min.

Step 2. Second strand cDNA synthesis: Prepare SS_Premix_{—}1 as follows: 2.9 μL RNase free water, 4.0 μL 17.5 mM MgCl₂, 0.4 μL 10 mM dNTPs, 2.5 μL 5 U/μL Klenow Fragment (exo−) (NEB), 0.2 μL, 2 U/μL RNase H (Invitrogen) for a total volume of 10 μL. Add 10 μL of the SS_Premix_{—}1 to each first strand reaction to make a final volume of 20 μL. Mix thoroughly and spin down, then incubate at 37° C. for 50 minutes. Inactivate the Klenow Fragment (exo⁻) at 70° C. for 10 min and keep at 4° C. for no longer than 10 min.

Step 3. IVT for cRNA amplification using Ambion MEGAscript T7 Kit: Add the following reagents to the 2nd strand synthesis reaction at room temperature according to the following order: 5 μL 75 mM ATP, 5 μL 75 mM CTP, 5 μL 75 mM GTP, 5 μL 75 mM UTP, 5 μL 10× reaction buffer, and 5 μL 10× enzyme mix for a total volume of 50 μL. Mix thoroughly after adding each reagent and spin briefly. Incubate at 37° C. for 16 hours.

Step 4. cRNA clean-up with Cleanup Module: Add 50 μL of RNase-free water to the above cRNA product. Follow the Cleanup Module protocol for cRNA purification. In the last step of cRNA purification, elute the product with 13 μL of RNase-free water. Remove 2 μL of the cRNA and measure the absorbance at 260 nm to determine the cRNA yield.

Step 5. Converting cRNA to first strand cDNA: Mix the cRNA (˜6 μg cRNA in 7 μL) and 1 μL 3 μg/μL random primers thoroughly in a 0.2 mL PCR tube. Spin briefly and incubate at 70° C. for 5 minutes and at 25° C. for 5 minutes. Prepare RT_Premix_{—}2 as follows: 4 μL 5× first strand buffer, 2 μL 0.1 M DTT, 1 μL 10 mM dNTP+dUTP mix, 1 μL 40 U/μL RNaseOUT, and 4 μL 200 U/μL SuperScript II, for a total volume of 12 μL. Add 12 μL of the RT_Premix_{—}2 to the denatured RNA and primer mixture to make a final volume of 20 μL. Mix thoroughly and spin briefly. Incubate at 25° C. for 5 minutes, then 42° C. for 1 hour and keep at 4° C. for no longer than 10 minutes.

Step 6. Second stranded cDNA Synthesis: Prepare SS_Premix_{—}2 as follows: 5.5 μL RNase free water, 8.0 μL 17.5 mM MgCl₂, 0.6 μL 10 mM dNTP+dUTP mix, 5.4 μL 6.2 U/μL E. coli DNA Polymerase (Invitrogen) and 0.5 μL 2 U/μL RNase H (Invitrogen), for a total volume of 20 μL. Add 20 μL of the SS_Premix_{—}2 to each first strand reaction to make a final volume of 40 μL. Mix thoroughly and spin down, then incubate at 37° C. for 40 minutes, at 75° C. for 10 min and keep at 4° C. for no longer than 10 min to proceed to the next step or freeze at −20° C.

Step 7. Double-stranded cDNA clean-up: Follow the Cleanup Module protocol to clean up the double stranded cDNA. In the last step of the double stranded cDNA purification, elute the product with 18 μL of Elution Buffer twice. Remove 2 μL of the cDNA eluate and measure the absorbance at 260 nm to determine the cDNA yield. Each tube contains about 8 μg to do fragmentation and labeling.

Step 8. Double stranded cDNA fragmentation: Prepare the following mix: 4.8 μL NEBuffer 4, 32 μL ds cDNA (8 μg), 4.0 μL UDG (2U/μL) (NEB) and 7.0 μL APE 1 (10U/μL) (NEB). Total volume is ˜48.0 μL. Spin briefly, incubate at 37° C. for 1 hr and inactivate the UDG at 93° C. for 1 minute, then keep at 4° C. Take 2 μL of the fragmented cDNA to check the average fragment size with RNA nano kit on an Agilent 2100 Bioanalyzer following the kit instruction. The desirable fragment size should be from 50 to 100 nt.

Step 9. Fragmented cDNA labeling: Prepare the labeling mix as follows: 16.8 μL 5×TdT Reaction buffer, 16.8 μL 25 mM CoCl₂, 1.2 μL 5 mM DLR-1a (Affymetrix), and 5.3 μL rTDT (400 U/μL) (Promega). Total Volume is about 40.0 μL. Add 40 μL of the labeling mix to 44 μL of the fragmented cDNA to make a final volume of 84 μL. Mix and spin briefly. Incubate at 37° C. for 60 minutes and keep at 4° C. Stop the reaction by adding 2 μL of 0.5M EDTA pH 8.0. Remove 14 μL for gel shift analysis.

Step 10. Hybridization: Prepare the Hybridization Mix as follows: 100 μL 2×MES Hybridization buffer, 3 μL 3 mM Control Oligo B2, 10 μL 20×RNA control, 2 μL 50 mg/μL BSA, acetelated, 2 μL 10 mg/μL Herring sperm DNA, and 14 μL 100% DMSO. Total volume is about 130 μL. Add 130 μL of the Hybridization Mix to 70 μL of the combined labeling reaction to make a final volume of 200 μL, mix well and denature at 99° C. for 10 minutes and keep at 50° C. for 5 minutes in a thermal cycler. Hybridize the 200 μL labeled cDNA to pre-wetted GeneChip probe array (U133A 2.0 arrays) at 50° C. for 16 hours. Follow the wash and scan procedures described in the GeneChip Expression Analysis Technical Manual.

### EXAMPLE 4

### Effect of dUTP:dTTP on Fragment Size

Fragment size may be controlled by varying the ratio of dUTP to dTTP in the cDNA synthesis reaction. A human heart sample was amplified and fragmented as described above varying the ratio of dUTP to dTTP. Ratios of 1:3, 1:5 and 1:8 were tested. 1:3 resulted in a peak at approximately 55 bases, 1:5 a peak at approximately 65 bases and 1:8 a peak at approximately 80 bases. The labeled fragments were hybridized to a U133A 2.0 array (Affymetrix) to determine average percent present (% P). Ratios of dUTP:dTTP of 1:3 and 1:5 gave average % P of between 50 and 55% while 1:8 gave an average % P of about 45%.

### EXAMPLE 5

### GeneChip® Whole Transcript (WT) Sense Target Labeling Assay

For additional detail regarding the method described in Example 5 see GENECHIP® Whole Transcript (WT) Sense Target Labeling Assay Manual available from Affymetrix (P/N 701880 Rev. 2) which is incorporated herein by reference in its entirety.

Step 1. rRNA reduction using RIBOMINUS™ Kit from Invitrogen. (A.) Mix Hybridization Buffer from Invitrogen kit with Betaine, 54 μL 5 M Betaine with 126 μL hybridization buffer. (B.) Mix 3 μL of total RNA with Poly-A RNA controls added, 0.8 μL RiboMinus Probe, 100 pmol/μL, and 20 μL hybridization buffer with Betaine. Incubate at 70° C. for 5 minutes and place tube on ice. (C.) Re-suspend the magnetic beads from the RIBOMINUS kit and pipette 50 μL of the bead suspension into a non-stick RNase-free tube. Place the tube on a magnetic stand and discard supernatant. Wash beads twice with 50 μL RNase-free water and a third wash with 50 μL Hybridization buffer with Betaine. Re-suspend the beads in 30 μL of Hybridization buffer with Betaine. Incubate at 37° C. for 1-2 minutes. Transfer the total RNA sample prepared in (B.) to the beads prepared in (C.) and incubate at 37° C. for 10 min. (flick once to mix after 5 min). Place on magnetic stand and transfer the supernatant to a new 1.5 mL non-stick RNase-free tube, leave on ice. Wash the beads with 50 μL of hybridization buffer with Betaine and incubate at 50° C. for 5 minutes. Collect supernatant and combine with the first supernatant for a total of about 100 μL.

Concentrate the supernatant by adding 350 μL cRNA binding buffer (with ethanol already added) to each sample. Vortex for 3 seconds. Add 250 μL 100% ethanol to each tube. Apply sample to cRNA spin column and centrifuge. Wash column with 500 μL of cRNA wash buffer. Wash column with 500 μl of 80% ethanol. Spin column with open cap for 5 minutes. Transfer column to a clean 1.5 mL collection tube and elute with 11 μL RNase-free water. The eluate is the rRNA reduced RNA sample.

Step 2. First-Cycle, First strand cDNA synthesis: Addition of T7-N₆ primers: Dilute the primers 1:5 with RNase-free water. Add 1 μL diluted primers to 4 μL rRNA reduced sample. Incubate at 70° C. for 5 min and at 4° C. for at least 2 min. Place on ice. The sequence of the primer may be that of (SEQ ID NO: 4). Then spin down to collect the sample. Prepare the Master Mix as follows: 2.0 μL 5×1^{st} strand buffer, 1.0 μL 0.1 M DTT, 0.5 μL 10 mM dNTP mix, 0.5 μL 40 U/μL RNase Inhibitor, and 1.0 μL 200 U/μL SuperScript II (Invitrogen) in a total volume of 5 μl. Add 5 μL of the Master Mix to the sample to make a final volume of 10 μL. Mix thoroughly, spin down, and incubate at 25° C. for 10 min., at 42° C. for 1 hour, at 70° C. for 10 min. then keep at 4° C. for 2 to 10 min.

Step 3. First-Cycle, Second strand cDNA synthesis: Mix 2 μL of 1M MgCl₂ with 112 μL RNase-free water to make up 17.5 mM MgCl₂ dilution. Prepare master mix as follows: 4.8 μL Rnase free water, 4.0 μL 17.5 mM MgCl₂, 0.4 μL 10 mM dNTP's, 0.6 μL DNA Polymerase 1, 0.2 μL, 2 U/μL RNase H (Invitrogen) for a total volume of 10 μL. Add 10 μL of the Master Mix to each sample to make a final volume of 20 μL. Mix thoroughly and spin down, then incubate at 16° C. for 120 minutes without heated lid, 75° C. for 10 minutes with heated lid and 4° C. fror 2-10 minutes.

Step 4. First-cycle, cRNA synthesis. GeneChip WT cDNA amplification kit: Prepare the master mix as follows (volumes are for 1 reaction): 5 μl 10×IVT buffer, 20 μl IVT NTP mix, and 5 μl IVT enzyme mix. Add 30 μl of the master mix to each sample and incubate at 37° C. for 16 hours.

Step 5. cRNA clean-up with Cleanup Module: Add 50 μL of RNase-free water to the product of step 4, bringing total to 100 μl. Add 350 μl of cRNA binding buffer to each tube and vortex. Add 250 μl of 100% Ethanol to each tube. Apply the sample to IVT cRNA spin column and centrifuge. Wash column with 500 μl cRNA wash buffer. Wash again with 500 μl of 80% ethanol. Spin column with open cap for 5 minutes. Transfer column to a clean 1.5 mL collection tube and elute with 12 μl RNase-free water. Quantitate the cRNA yield.

Step 6. Second-cycle, first-strand cDNA synthesis: Add 1.5 μl of Random Primers to 8-10 μg of the cRNA sample from step 5 and bring to 8 μl with RNase-free water. Incubate at 70° C. for 5 minutes, at 25° C. for 5 minutes and at least 2 minutes at 4° C. Prepare Master Mix as follows (volume for 1 reaction): 4 μL 5×1^{st} strand buffer, 2 μL 0.1 M DTT, 1.25 μL 10 mM dNTP mix+dUTP (10 mM each of dATP, dCTP and dGTP, 8 mM dTTP and 2 mM dUTP), and 4.75 μL 200 U/μL SuperScript II, for a total volume of 12 μL. Add 12 μL of the Master Mix to the RNA and primer mixture to make a final volume of 20 μL. Mix thoroughly and spin briefly. Incubate at 25° C. for 5 minutes, then 42° C. for 90 minutes, then 70° C. for 10 minutes and 4° C. for at least 2 minutes.

Step 7. Hydrolysis of cRNA and Cleanup of Single-stranded cDNA: Add 1 μl RNase H to each sample and incubate at 37° C. for 45 minutes, 95° C. for 5 minutes and 4° C. for 2 minutes. Add 80 μl RNase-free water to each sample. Add 370 μl of cDNA binding buffer and vortex. Apply sample to cDNA spin column and centrifuge. Wash column with 750 μl of cDNA wash buffer (with 100% ethanol already added). Spin column with open cap for 5 minutes. Transfer colun to a clean 1.5 ml collection tube and elute with 15 μl of cDNA elution buffer. Elute again with 15 μl of cDNA elution buffer. Combine the eluate, mix well, and quantitate the single-stranded DNA yield.

Step 8. Fragmentation of Single-Stranded DNA: Prepare the following fragmentation cocktail (volumes for 1 reaction): 4.8 μL 10×cDNA fragmentation buffer, 5.5 μg single stranded DNA, 1.0 μL UDG (10 U/μL), 1.0 μL APE 1 (1000 U/μL) (NEB) and RNase free water to a total volume of 48.0 μL. Spin briefly, incubate at 37° C. for 1 hr and inactivate the UDG at 93° C. for 2 minute, then at 4° C. for at least 2 minutes. Transfer 45 μl to a new tube and use the rest to analyze the size of the fragments using a Bioanalyzer (Agilent). The range in peak size of the fragmented samples should be approximately 40-70 bp.

Step 9. Labeling of fragmented single-stranded DNA: Prepare the Labeling mix as follows: 12 μL 5×TdT Reaction buffer, 1 μL DLR-1a (Affymetrix), 5 mM, and 2 μL TdT and 45 μL of the fragmented DNA to make a final volume of 60 μL. Mix and spin briefly. Incubate at 37° C. for 60 minutes, 70° C. for 10 minutes and 4° C. for at least 2 minutes.

Step 10. Hybridization: Prepare the Hybridization Mix as follows: 110 μL 2×MES Hybridization buffer, 3.7 μL Control Oligo B2 (final is 50 pM), 11 μL Eukaryotic Hybridization Contols (bioB, bioC, bioD, cre)(heat at 65° C.), 2.2 μL 50 mg/μL BSA, acetelated, 2.2 μL 10 mg/μL Herring sperm DNA, and 15.4 μL 100% DMSO, ˜60 μl fragmented and labeled DNA target and RNase free water to a final volume of 220 μl. Mix well and denature at 99° C. for 5 minutes and cool to 45° C. for 5 minutes in a thermal cycler, quick centrifuge. Add 200 μL labeled cDNA to an equilibrated GeneChip Exon array at 50° C. for 16 hours. Place the array in 45° C. hybridization oven at 60 rpm for 16 hours.

Step 11. Washing and Staining: Prepare the staining reagents: 2× stain buffer, 10 mg/ml goat IgG, 0.5 mg/ml biotinylated antibody, 50 mg/ml BSA, wash buffer A, wash buffer B and 1× array holding buffer. Prepare SAPE stain solutionP: 300 μl 2× stain buffer, 24 μl 50 mg/ml BSA, 6 μl 1 mg/ml SAPE, and 270 μl water for final volume of 600 μl. Prepare antibody solution as follows: 300 μl 2× stain buffer, 24 μl 50 mg/ml BSA, 6 μl 10 mg/ml Goat IgG stock, 3.6 μl 0.5 mg/ml biotinylated antibody and 266.4 μl water for final volume of 600 μl. Follow the wash and scan procedures described in the GENECHIP® Whole Transcript (WT) Sense Target Labeling Assay Manual available from Affymetrix (P/N 701880 Rev. 2) which is incorporated herein by reference in its entirety.

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

We claim:

1. A method for obtaining a nucleic acid amplification product comprising labeled cDNA fragments from a nucleic acid sample containing RNA, the method comprising:
a.) providing a first nucleic acid sample comprising RNA;
b.) amplifying the first nucleic acid sample to obtain a second nucleic acid sample comprising single stranded cDNA, wherein said single stranded cDNA contains uracil;
c.) cleaving the single stranded cDNA by a method comprising incubating the single stranded cDNA in a reaction with UDG and an AP endonuclease, wherein said AP endonuclease is active on single stranded cDNA, to generate single-stranded cDNA fragments; and
d.) labeling said single stranded cDNA fragments in a reaction comprising TdT and at least one labeled nucleotide to obtain labeled cDNA fragments.

2. The method of claim 1 wherein step b.) comprises:
synthesizing first strand cDNA from said RNA by reverse transcription using primers comprising a random portion and an RNA polymerase promoter portion;
synthesizing second strand cDNA to obtain double stranded cDNA comprising an RNA polymerase promoter;
generating cRNA by in vitro transcription of said double stranded cDNA; and
generating single-stranded cDNA from said cRNA by reverse transcription using random primers in the presence of dUTP followed by removal of the cRNA strand by a method selected from the group consisting of RNase H treatment and alkali treatment.

3. The method of claim 2 wherein said second strand cDNA is synthesized in a reaction comprising E. coli DNA polymerase I and RNase H.

4. The method of claim 1 wherein the reaction of step c.) has 150 to 200 units of AP endonuclease for each microgram of single stranded cDNA.

5. The method of claim 4 wherein the reaction contains 5 to 6 micrograms of single stranded cDNA.

6. The method of claim 4 wherein the volume of the reaction of step c.) is between 35 and 60 microliters.

7. The method of claim 1, wherein said uracil containing cDNA is obtained by reverse transcribing cRNA in the presence of a first amount of dTTP and a second amount of dUTP, wherein the ratio of dTTP to dUTP is about 4 to 1.

8. The method of claim 1, wherein said uracil containing cDNA is obtained by reverse transcribing cRNA in the presence of a first amount of dTTP and a second amount of dUTP, wherein the ratio of dTTP to dUTP is about 8 to 1.

9. The method of claim 1, wherein said uracil containing cDNA is obtained by reverse transcribing cRNA in the presence of a first amount of dTTP and a second amount of dUTP, wherein the ratio of dTTP to dUTP is about 5 to 1.

10. The method of claim 1, wherein said uracil containing cDNA is obtained by reverse transcribing cRNA in the presence of a first amount of dTTP and a second amount of dUTP, wherein the ratio of dTTP to dUTP is about 3 to 1.

11. The method of claim 1, wherein the average size of the single stranded cDNA fragments is about 40 to 150 bases in length.

12. The method of claim 1, wherein the average size of the single stranded cDNA fragments is 40 to 70 bases in length.

13. The method of claim 1, wherein the AP endonuclease is APE 1.

14. A method for analyzing the expression of a plurality of genes in a sample, said method comprising:
a.) obtaining a first nucleic acid sample comprising mRNA from said sample;
b.) generating a second nucleic acid sample comprising cDNA by a method comprising mixing said first nucleic acid sample in a reaction comprising a primer including a 3′ portion comprising random sequence and a 5′ portion including an RNA polymerase promoter sequence and a reverse transcriptase;
c.) generating a third nucleic acid sample comprising second strand cDNA by a method comprising mixing said second nucleic acid sample in a reaction comprising RNase H and a DNA polymerase:
d.) generating a fourth nucleic acid sample comprising cRNA by a method comprising mixing said third nucleic acid sample with an RNA polymerase;
e.) generating a fifth nucleic acid sample comprising first strand cDNA by a method comprising mixing said fourth nucleic acid sample with random primers, a reverse transcriptase, dTTP, dGTP, dCTP, dATP and dUTP;
f.) generating a sixth nucleic acid sample comprising sense orientation single stranded cDNA by a method comprising mixing said fifth nucleic acid sample in a reaction comprising RNase H;
g.) fragmenting said sixth nucleic acid sample in a reaction comprising UDG and APE 1 to obtain single stranded cDNA fragments;
h.) labeling said single stranded cDNA fragments in a reaction comprising terminal transferase and a labeled nucleotide to obtain labeled fragments;
i.) hybridizing said labeled fragments to an array comprising more than 100,000 probes to generate a hybridization pattern; and
j.) analyzing said hybridization pattern.

15. The method of claim 14 wherein APE 1 is added so that there is more than 150 units of APE 1 for each microgram of single stranded cDNA.

16. The method of claim 14 wherein the ratio of dTTP to dUTP in step e.) is about 4 to 1.

17. The method of claim 14 further comprising adding a control oligonucleotide to the first, second, third, fourth, fifth or sixth nucleic acid sample,
wherein the control oligonucleotide comprises a 5′ first region and a 3′ second region wherein said first and second regions are separated by at least one uracil or abasic site, and wherein said array comprises probes to said first region and probes to said second region, and wherein said control oligonucleotide is modified at the 3′ end to block labeling and extension; and
analyzing the hybridization pattern to determine the efficiency of fragmention of the control oligonucleotide, wherein labeling and detection of the first region is indicative of fragmentation.

18. A method to determine the efficiency of fragmentation of a complex nucleic acid sample by a UDG and APE 1 mediated fragmentation process comprising:
a.) obtaining a control oligonucleotide wherein said control oligonucleotide comprises a 5′ first region and a 3′ second region separated by at least one uracil or at least one abasic position, wherein the 3′ end of the control oligonucleotide is not a substrate for terminal labeling by TdT;
b.) adding an aliquot of said control oligonucleotide to said complex nucleic acid sample to generate a mixture;
c.) treating said mixture with a UDG activity and an APE 1 activity to obtain fragments, wherein said control oligonucleotide is cleaved into a first fragment comprising said first region and a second fragment comprising said second region;
d.) labeling at least some of the products of step c.) in a reaction comprising TdT;
e.) hybridizing at least some of the products of step d.) to a microarray, wherein the microarray comprises probes for the first region of the control oligonucleotide; and
f.) analyzing the hybridization pattern to determine the efficiency of fragmentation of the control oligonucleotide.

19. The method of claim 18, wherein the control oligonucleotide is double stranded.

20. The method of claim 18, wherein the complex nucleic acid sample comprises primarily double-stranded DNA and the control oligonucleotide is double stranded.

21. The method of claim 18, wherein the complex nucleic acid sample comprises primarily single-stranded DNA and the control oligonucleotide is single stranded.

22. The method of claim 18, wherein the complex nucleic acid sample comprises a mixture of double and single stranded DNA, which may be present at an unknown ratio.

23. A control oligonucleotide comprising from the 5′ end, a first region, a cleavage position, a second region and a 3′ terminal modification blocking 3′ extension or labeling of the control oligonucleotide at its 3′ end.

24. The control oligonucleotide of claim 23 wherein the control oligonucleotide comprises a region of at least 10 bases that is double stranded.

25. The control oligonucleotide of claim 23 wherein the control oligonucleotide is completely single stranded.

26. The control oligonucleotide of claim 23 wherein said cleavage position comprises 1 to 5 uracils.

27. The control oligonucleotide of claim 23 wherein said cleavage position is at least one abasic position.

28. The control oligonucleotide of claim 23, wherein the modification comprises a 3′ terminal phosphate group.

29. The control oligonucleotide of claim 23, wherein the modification comprises a modified base.

30. The control oligonucleotide of claim 23, wherein the modification comprises an amino group.

31. The control oligonucleotide of claim 23, wherein the modification comprises a 3′ deoxy base.

32. The control oligonucleotide of claim 23, wherein the modification comprises a 3′-3′ reverse linkage at the terminal end.

33. A method for obtaining a nucleic acid amplification product comprising labeled cDNA fragments from a nucleic acid sample containing RNA, the method comprising:
a.) providing a first nucleic acid sample comprising RNA;
b.) synthesizing single-stranded cDNA containing uracil from said RNA in a reaction comprising a reverse transcriptase, random primers, dUTP, dGTP, dCTP, dATP and dTTP;
c.) cleaving the single stranded cDNA by a method comprising incubating the single stranded cDNA in a reaction with UDG and an AP endonuclease, wherein said AP endonuclease is active on single stranded cDNA, to generate single-stranded cDNA fragments; and
d.) labeling said single stranded cDNA fragments in a reaction comprising TdT and at least one labeled nucleotide to obtain labeled cDNA fragments.

34. The method of claim 33 wherein the labeled nucleotide is biotinylated.

35. The method of claim 33 wherein the AP endonuclease is APE 1.

36. The method of claim 33 wherein the ratio of dTTP to dUTP is about 4 to 1.

37. The method of claim 33 wherein following step b.) the RNA is removed by treatment with RNase H or alkali.

38. A kit comprising a solution of T7-N₆ primers, buffer, DTT, dGTP, dCTP, dATP, a solution of dTTP and dUTP, an RNase inhibitor, a reverse transcriptase, a DNA polymerase, APE 1, and random primers, wherein the ratio of dTTP to dUTP in the solution of dTTP and dUTP is about 4 to 1.

39. The kit of claim 39 further comprising a solution of random primers.

40. The kit of claim 38 wherein the DNA polymerase is E. coli DNA polymerase.

41. The kit of claim 38 wherein the DNA polymerase is Klenow (exo−).
